(19) United States
(12) Patent Application Publication
(10) Pub. No.: US 2025/0221656 A1
(43) Pub. Date: Jul. 10, 2025

(54) EYE TRACKING-BASED DETECTION AND CORRELATION WITH NEUROLOGICAL CONDITION

(71) Applicant: Stardots AB, Uppsala (SE)

(72) Inventors: Daniel Petrini, Uppsala (SE); Fredrik Olsson, Uppsala (SE); Alexander Medvedev, Täby (SE)

(21) Appl. No.: 19/014,803

(22) Filed: Jan. 9, 2025

Related U.S. Application Data
(60) Provisional application No. 63/619,030, filed on Jan. 9, 2024.

(51) Int. Cl. A61B 5/00; A61B 5/11; G16H 20/00; G16H 50/20

(52) U.S. Cl. CPC A61B 5/4058; A61B 5/1103; A61B 5/1126; A61B 5/4842; A61B 5/742; A61B 5/7475; G16H 20/00; G16H 50/20

(57) ABSTRACT

A method of managing a neurological condition includes displaying a test target. An image capture device detects movement of a patient eye with respect to the test target. Data relating to the movement of the patient eye is received as an input to a machine learning model. The data relating to the movement of the patient eye with respect to the test target is correlated to a neurological condition, and an objective output information is produced by the machine learning model based on the correlation of the data to the neurological condition indicating a level of progression of the neurological condition.

### CROSS REFERENCE TO RELATED APPLICATIONS

This application claims the benefit of U.S. Provisional Patent Application No. 63/619,030, filed Jan. 9, 2024, the contents of which are incorporated by reference herein in its entirety as if fully set forth.

### FIELD

Illustrative embodiments of the invention generally relate to medical devices and, more particularly, various embodiments of the invention relate to tracking eye movement to manage patient treatment.

### BACKGROUND

Medical diagnostics and treatment often require testing with subjective criteria. For example, a medical professional may ask patients about the presence of various symptoms or ask patients to rate symptoms on an arbitrary scale.

### SUMMARY OF VARIOUS EMBODIMENTS

In accordance with one embodiment of the invention, a method of managing a neurological condition includes displaying a test target. An image capture device detects movement of a patient eye with respect to the test target. Data relating to the movement of the patient eye is received as an input to a machine learning model. The data relating to the movement of the patient eye with respect to the test target is correlated to a neurological condition, and an objective output information is produced by the machine learning model based on the correlation of the data to the neurological condition indicating a level of progression of the neurological condition.

In some embodiments, the detecting movement is performed during one or more eye movement tests.

In some embodiments, the one or more eye movement tests comprises one or more of a fixation test or a smooth pursuit test.

In some embodiments, the fixation test further comprises displaying the test target at one or more locations and detecting a latency, duration, peak amplitude, or peak velocity of the movement of the patient eye.

In some embodiments, the method further includes detecting a catch-up saccade of the movement of the patient eye.

In some embodiments, the smooth pursuit test comprises moving the test target along a trajectory on the display and detecting the movement of the patient eye following the trajectory.

In some embodiments, the method further includes training a machine learning model using the data relating to the movement of the patient eye during the eye movement test.

In some embodiments, the method further includes outputting a patient treatment parameter based upon the correlation of the data relating to the movement of the patient eye to a neurological condition.

In some embodiments, the method further includes planning a treatment for the neurological disorder based on the correlation of the data to the neurological condition indicating the level of progression of the neurological condition.

In accordance with one embodiment of the invention, a system includes an image capture device configured to detect movement of a patient eye with respect to a displayed test target, and a processor in communication with the image capture device. The processor is configured to receive, from the image capture device, data relating to the movement of the patient eye as an input to a machine learning model, correlate the data relating to the movement of the patient eye with respect to the test target to a neurological condition, and produce objective output information by the machine learning model based on the correlation of the data to the neurological condition indicating a level of progression of the neurological condition.

In some embodiments, the image capture device detects movement during one or more eye movement tests.

In some embodiments, the one or more eye movement tests comprises one or more of a fixation test or a smooth pursuit test.

In some embodiments, the fixation test further comprises displaying the test target on the display at one or more locations on the display and detecting a latency, duration, peak amplitude, or peak velocity of the movement of the patient eye.

In some embodiments, the image capture device detects a catch-up saccade of the movement of the patient eye.

In some embodiments, the smooth pursuit test comprises moving the test target along a trajectory on the display and detecting the movement of the patient eye following the trajectory.

In some embodiments, the processor is further configured to train a machine learning model using the data relating to the movement of the patient eye during the eye movement test.

In some embodiments, the processor is further configured to output a patient treatment parameter based upon the correlation of the data relating to the movement of the patient eye to a neurological condition.

In some embodiments, the processor is further configured to output a plan for a treatment for the neurological disorder based on the correlation of the data to the neurological condition indicating the level of progression of the neurological condition.

In accordance with one embodiment of the invention, a computer program product for use on a computer system includes a tangible, non-transient computer usable medium having computer readable program code thereon, the computer readable program code includes program code for displaying a test target, program code for receiving data relating to a movement of a patient eye with respect to the test target as an input to a machine learning model, program code for correlating the data relating to the movement of the patient eye with respect to the test target to a neurological condition, and program code for producing objective output information by the machine learning model based on the correlation of the data to the neurological condition indicating a level of progression of the neurological condition.

In some embodiments, the non-transitory computer-readable medium further includes outputting, by the processor, a patient treatment parameter based upon the correlation of the data relating to the movement of the patient eye to a neurological condition.

Illustrative embodiments of the invention are implemented as a computer program product having a computer usable medium with computer readable program code thereon. The computer readable code may be read and utilized by a computer system in accordance with conventional processes.

### BRIEF DESCRIPTION OF THE DRAWINGS

Those skilled in the art should more fully appreciate advantages of various embodiments of the invention from the following “Description of Illustrative Embodiments,” discussed with reference to the drawings summarized immediately below.

FIG. 1 schematically shows an example patient analysis system in accordance with various embodiments.

FIG. 2 shows an example patient test environment in accordance with various embodiments.

FIG. 3 is a flow diagram of an example method 300 for eye tracking-based diagnostics and treatment in accordance with an illustrative embodiment.

FIG. 4 shows an example graphical representation of fixation points of a fixation test administered on the patient device display in accordance with various embodiments.

FIG. 5 shows an example graphical representation of detected gaze points of a fixation test in accordance with various embodiments.

FIG. 6 shows an example graphical representation of a smooth pursuit trajectory of a smooth pursuit test administered on the patient device display in accordance with various embodiments.

FIG. 7 schematically shows a machine learning model for patient diagnostics in accordance with various embodiments.

FIG. 8 shows an example modeling pipeline for complex valued VL modeling of smooth pursuit in accordance with various embodiments.

FIG. 9 is a block diagram schematically showing a computing device in accordance with various embodiments.

### DESCRIPTION OF ILLUSTRATIVE EMBODIMENTS

In illustrative embodiments, a patient analysis system determines a diagnosis or treatment for a neurological condition (e.g., Parkinson's Disease or Alzheimer's Disease) based on eye movement of a patient captured during a series of tests. The tests May capture eye movement during periods of fixating on a point or tracking a moving point on a screen. Test data may then be used to determine parameters, including system models, for describing the patient's performance. Parameters may correspond to fixating on a point, transitioning from fixed point to fixed point, or catching up to a moving point, among other things. The derived parameters may then be used to diagnose and/or treat the patient. In some embodiments, the diagnosis or treatment may correspond to a neurological disease, such as, for example, Parkinson's Disease or Alzheimer's Disease, among other things. In some embodiments, a test administrator may use the patient analysis system to modify the administered tests, select inputs from the derived parameters and systems models in a user interface, and generate or use a model for diagnosing/treating a disease. Details of illustrative embodiments are discussed below.

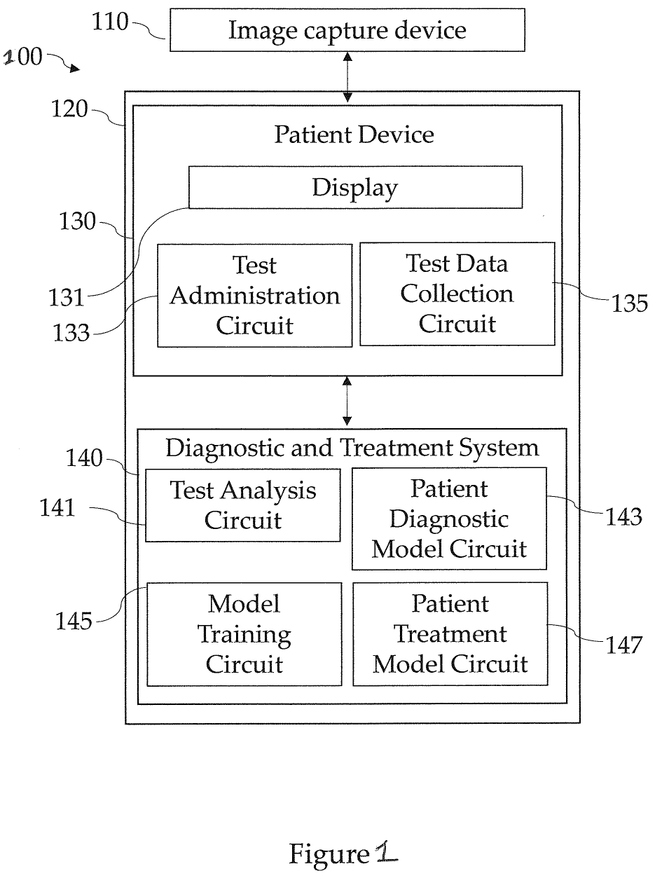

FIG. 1 is a box diagram showing a patient analysis system 100 in accordance with various embodiments. The patient analysis system 100 has an image capture device 110 for collecting data corresponding to eye movement of a patient. In various embodiments, the image capture device 110 is an eye tracking camera.

In various embodiments, the eye movement captured by the image capture device 100 may include fixations (e.g., gazing upon a stationary target), saccades (e.g., quick eye movement used to move from one fixation to another to track a target moving rapidly or instantaneously), and/or smooth pursuit (e.g., tracking a target that moves smoothly, such as velocities up to 30°/s of visual angle). In various embodiments, the image capture device 100 may also collect data regarding the pupil dilation, blinking rate, or eye openness of the patient.

The patient analysis system 100 also includes a computer system 120 which may include devices proximate or remote from the image capture device 110. For example, the illustrated computer system 120 has a patient device 130 configured to administer tests to the patient and collect the test data. The patient device 130 includes a display 131 for outputting a visual representation of the test to the patient.

The patient device 130 also has a test administration circuit 133 for providing each test to the display 131. The test administration circuit 133 may provide a series of tests, described in further detail below.

The patient device 130 also has a test data collection circuit 135 for receiving the data collected during the test and providing the test data for further analysis. In various embodiments, the data collection circuit 135 may determine, for one or both eyes, 2D gaze position (position on screen where patient is looking); 3D gaze vectors (patient's eye gaze direction, distance between eye and screen position); the 3D eye position (location of the patient's eyes in the camera space). The data collection circuit 135 may also determine pupil size (dilation or change in dilation) or eyelid openness (distance between eyelids), among other things. The data collection circuit 135 may determine data by receiving the data from the image capture device 110 or processing data received from the image capture device 110. In some embodiments, the data is collected at a rate of at least 120 Hz.

The computer system 120 may also include a diagnostic and treatment system 140 for determining patient test parameters from the collected data and generating diagnostic/treatment models using the derived patient test parameters.

In various embodiments, the patient diagnostics and treatment system 140 has a test analysis circuit 141 for determining patient test parameters from the data determined by the data collection circuit 135. The patient diagnostic and treatment system 140 may also have a model training circuit 145 for generating machine learning models for diagnosis or treatment, where the machine learning models use patient test parameters as inputs. The patient diagnostic and treatment system 140 may also include a patient diagnostic model circuit 143 for using the generated machine learning models to diagnose a patient using patient test parameters. The patient diagnostic and treatment system 140 may also include a patient diagnostic model circuit 147 for using the generated machine learning model to treat a patient using the patient test parameters.

It should be appreciated that any of the features of the patient analysis system 100 may also be present in the other embodiments disclosed herein.

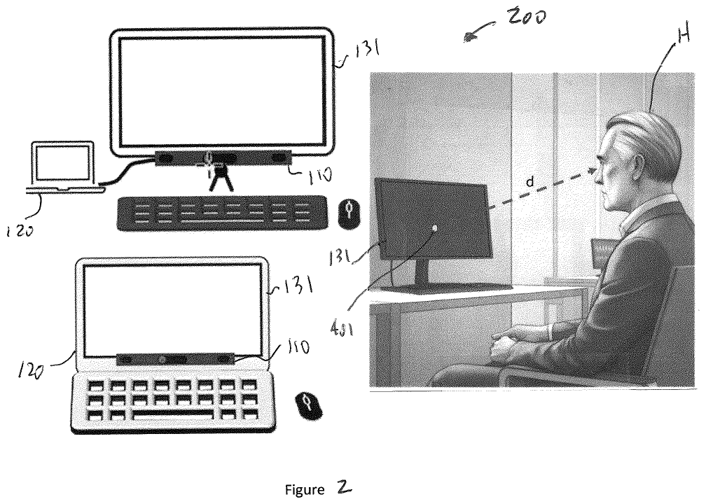

FIG. 2 shows an example patient test environment 200 in accordance with various embodiments. As shown in FIG. 2, a human ‘H’ to be tested may be seated in front of the display 131 at a distance ‘d’ observing a fixation point 401. In various embodiments, the distance d may be 65 centimeters (cm). Also, as shown in FIG. 2, in various embodiments, the image capture device 110 may be placed on a display 131 that is separate from the computer system 120 or may be placed on a display 131 that is integrated into the computer system 120 (e.g., a laptop).

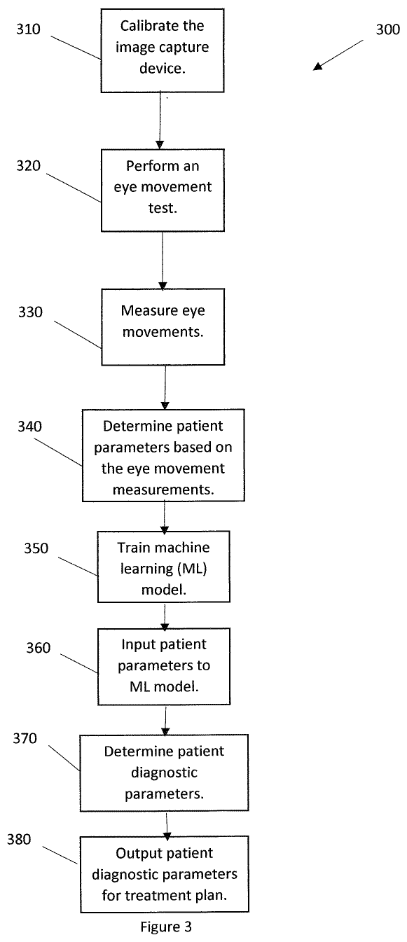

FIG. 3 is a flow diagram of an example method 300 for eye tracking-based diagnostics and treatment in accordance with an illustrative embodiment. In various embodiments, the method, or process, 300 may be performed using components described above in FIG. 1 and FIG. 2. In some embodiments, the functionalities may be performed by separate cloud devices or user devices of the computer system 120. In some embodiments, all functionalities may be performed by the same device. It shall be further appreciated that a number of variations and modifications to the method 300 are contemplated including, for example, the omission of one or more operations of the method 300, the addition of further conditionals and operations, or the reorganization or separation of operations and conditionals into separate processes.

In various embodiments, for example, as described above, the display 131 may be utilized to display points that are intended for the human H patient's eye to view, fixate on, and/or follow. The image capture device 110 may therefore capture the eye movement or fixation of the eye during one or more tests.

In order to accurately perform measurements, the image capture device 110 may require calibration so that when a test is being performed, the image capture device 110 captures accurate eye movements.

Accordingly, at operation 310, the image device is calibrated. In various embodiments, the calibration includes a sequence that displays a number (e.g., 5) of fixation points located centrally on display 131 (i.e., the center of the screen), then in each corner of a bounding box displayed on the screen that limits the stimulus movement. This sequence mimics the 5-point calibration sequence used for hardware calibration of the image capture device 110 (e.g., an eyetracker camera) itself. It is used to validate and further refine the calibration to improve the accuracy and precision of the subsequent measurements.

At operation 320, an eye movement test is performed. In various embodiments, the eye movement test may be one or more of a fixation test and/or a smooth pursuit test to measure eye movements. For example, a fixation test may test the ability of an eye's gaze to remain focused on a stationary target. Further, a saccade is a rapid eye movement used to move from one fixation to another or to track a target that moves rapidly or instantaneously that may be captured by the image capture device 110 during a test. In an example smooth pursuit test, the gaze is used to track a target that moves smoothly (e.g., for velocities up to 30°/s of visual angle) and measured. In a pupil dilation test, the pupil expands or contracts, typically to adapt to light conditions in the environment, and such expansion may be measured.

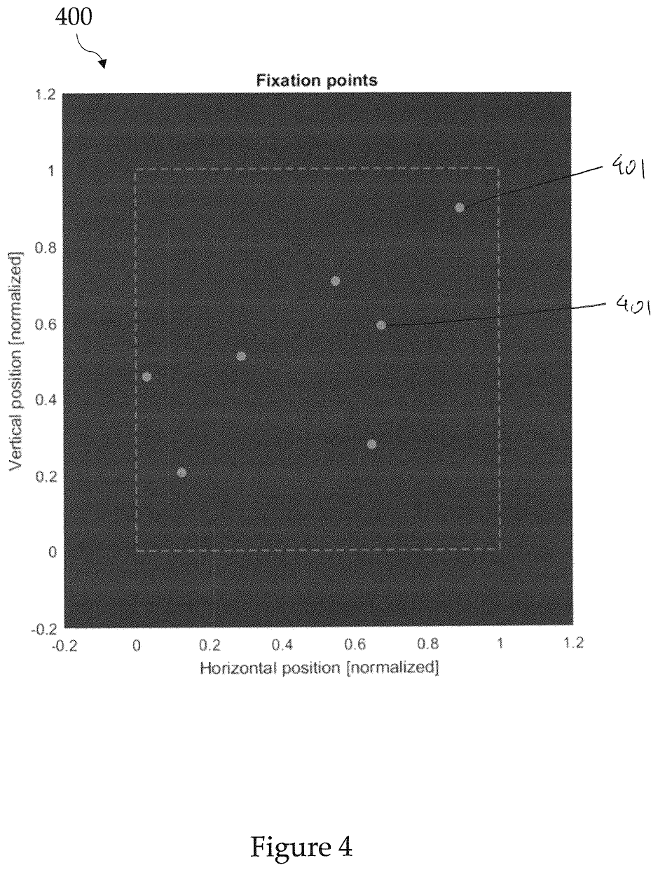

For a fixation test, a number (e.g., 7) of fixation points may be displayed randomly within the bounding box described above. FIG. 4 shows an example graphical representation 400 of fixation points (e.g., designated 401) of a fixation test administered on the patient device display in accordance with various embodiments. In various embodiments, the fixation point 401 may be a circle (e.g., 0.5 cm in diameter). For purposes of example, only two of the fixation points 401 are labeled. As described, the bounding box includes the area within the dashed line forming a square in the example shown in FIG. 4. In various embodiments, the fixation points 401 are targets that move from position to position while the image capture device 110 captures the movement of the eye following the fixation points 401 from location to location on the display 131.

Also, the fixation test captures saccade movements (described above) as well as the fixations, as the stimulus target (fixation point) makes instantaneous movements to its next location.

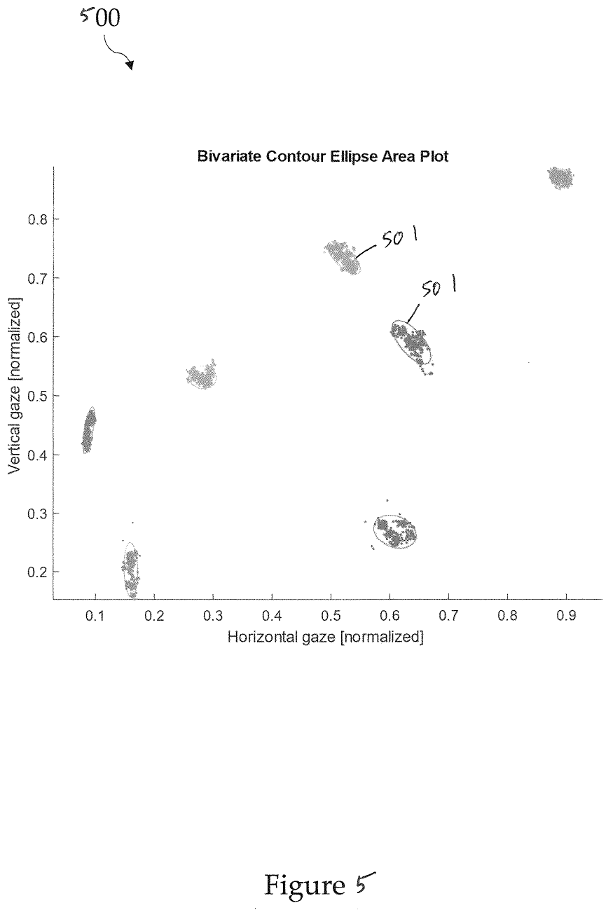

FIG. 5 shows an example graphical representation 500 of detected gaze points 501 of a fixation test in accordance with various embodiments. For purposes of example, only two of gaze points 501 are labeled. Although further detail is provided below, each of the gaze points 501 is shown bounded by bivariate contour ellipse.

In various embodiments, a smooth pursuit test displays a target following a continuous movement trajectory designed to be tracked by smooth pursuit of an eye. In order to allow for identification of both linear and nonlinear smooth pursuit dynamics, in various embodiments the target's movement may have a flat power spectrum up to 1.5 Hz as higher frequency movements may contain velocities outside the range of typical smooth pursuit for the human eye.

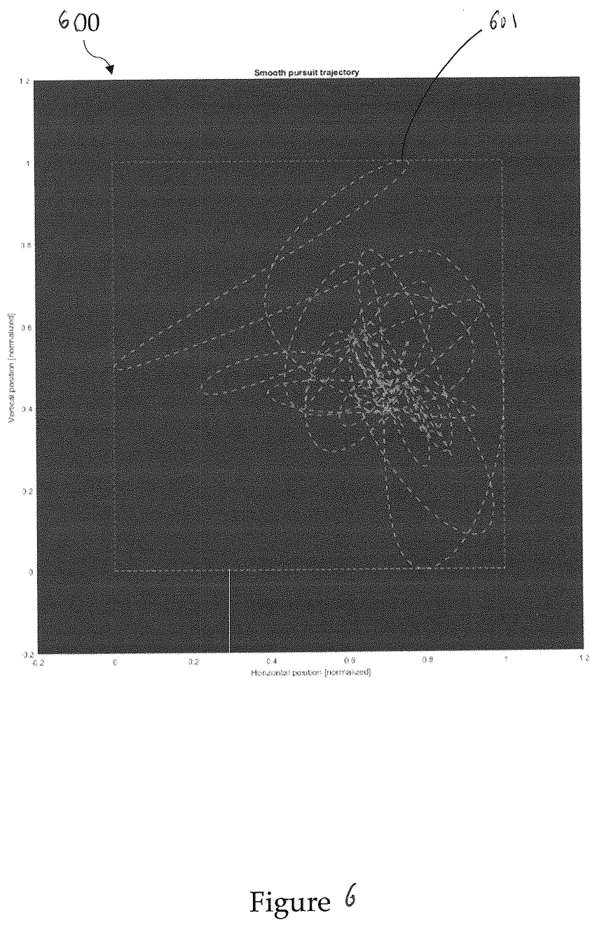

FIG. 6 shows an example graphical representation 600 of a smooth pursuit trajectory 601 of a smooth pursuit test administered on the patient device display in accordance with various embodiments. As shown in FIG. 6, the smooth pursuit trajectory 601 is a dashed line which indicates the movement of a target point along a path for the eye to follow/track.

At operation 330, the eye movements are measured. In various embodiments, for a fixation test, for example, eye fixations may be detected using a velocity threshold fixation filter (e.g., with a threshold of 60°/s). All fixations that are detected in the time interval where the visual target is in one location are grouped together into a set of fixation points such as the groupings 501 shown in FIG. 5.

In various embodiments, data points where the eyetracker has recorded valid eye detections for both left and right eyes are considered. Each set of fixation points is then passed through an outlier rejection algorithm that removes all points in a set that is outside three scaled median absolute deviations (MAD) from the median, where the scaled MAD is defined as:

$$MAD = c \times median ( \vert {A}_{i} - median ( A ) \vert ) ,$$

where A is a vector of N scalar observations and i=1, 2, . . . , N. The scale factor may be calculated in accordance with

$$c = - \frac{1}{\sqrt{2} erf inv ( \frac{2}{3} )} ,$$

where erfinv(·) denoted the inverse error function.

As mentioned above, A bivariate ellipse is fitted to cover a proportion of the remaining gaze points in each fixation set 501. In various embodiments, the bivariate ellipse contour area (BECA) is used to quantify the stability of the fixations, where a larger area corresponds to a less stable fixation. The BCEA may be computed in accordance with the following:

$$BCEA = 2 k π {σ}_{H} {{σ}_{V} ( 1 - ρ )}^{1 / 2} ,$$

where σ_{H} and σ_{V} are the standard deviations of the gaze points along the horizontal and vertical meridians, respectively, and ρ is the product-moment correlation of the horizontal and vertical components. The parameter k depends on the proportion of gaze points to be covered by the ellipse, where the proportion is given by the following:

$$P = 1 - {e}^{- k} .$$

The example in FIG. 5 shows a setting for P=0.65 which yields k=1.0498. Since the gaze coordinates are normalized to the area of the stimulus bounding box, BCEA may be obtained in cm².

Further, as mentioned above, saccade movements are captured during the above tests. For example, in various embodiments, the saccades are detected using an adaptive acceleration threshold algorithm. The detection algorithm may use angular accelerations derived from recorded 3D gaze and eye position data. The instantaneous gaze angle is computed as:

$${α}_{i} = {cos}^{- 1} \frac{{v}_{i - 1} \cdot {v}_{i}}{‖ {v}_{i - 1} ‖ ‖ {v}_{i} ‖} , i ∈ [ 2 , N ] ,$$

where N is the number of data points, vᵢ=pᵢ−oᵢ∈ [symbol] ³, is the gaze vector with origin in the eye positions oᵢ pointing at the gaze positions pᵢ. The instantaneous gaze angles αᵢ thus represents the angular shift between two subsequent measurements of the gaze vectors. Through two filtering operations, the angular velocities \dot{α}ₗ and angular accelerations \ddot{α}ₗ are obtained in units of °/s and °/s², respectively, are obtained.

In various embodiments, the saccade detection detects two peaks in the angular acceleration signal, with opposite sign that occur within a time interval corresponding to the maximum detectable saccade duration. This acceleration profile captures the rapid acceleration and subsequent deceleration that is typical for saccades.

Detecting the acceleration peaks uses an adaptive acceleration threshold may be performed in accordance with the following:

$$A C {C}_{th , adapt} = A C {C}_{t h , b a s e} + A C {C}_{R M S} ,$$

where the base threshold ACC_{th,base} is chosen differently depending on the application and type of saccades that should be detected, but typically in the range of 500-4000 m/s². The term ACC_{RMS} is the root mean square of \ddot{α}ₗ calculated for a centered window of, for example, 7 samples.

At operation 340, patient parameters are determined based on the eye movement measurements. For example, different saccades may be captured and different saccade parameters may be extracted. For example, fixation saccades are the saccades that occur when the fixation target moves instantaneously from one location to another. In various embodiments, they are the saccade of largest amplitude that occurs between the time when the fixation target moves until a fixation has been detected. The following saccade parameters may be extracted: latency (e.g., the time it takes from a movement of the fixation target until a saccade is detected), duration (e.g., the time it takes from a saccade is detected until a fixation is detected), peak amplitude (e.g., the largest change in visual angle occurring within the saccade duration time interval), and/or peak velocity (e.g., the highest velocity in terms of visual angle per time unit that occurs within the saccade duration time interval).

In another example, catch-up saccades are saccades that may occur when a person is trying to track a target with smooth pursuit, but is unable to keep up with the target. When a person's gaze falls too far behind the target, the eyes may instead use saccadic movements to try to keep up with the target. These catch-up saccades typically happen intermittently during smooth pursuit, with the eyes switching between smooth pursuit and saccadic movements automatically.

In various embodiments, catch-up saccades typically may have lower amplitudes and velocities than the fixation saccades and therefore may require a lower base threshold to be detected. Catch-up saccades may be more difficult to detect since they occur during dynamic smooth pursuit movement instead of between static fixations. The following catch-up saccade parameter may be quantified during an eye test described above: a number/frequency of catch up saccades during the smooth pursuit test, a duration, peak amplitudes, and/or peak velocities.

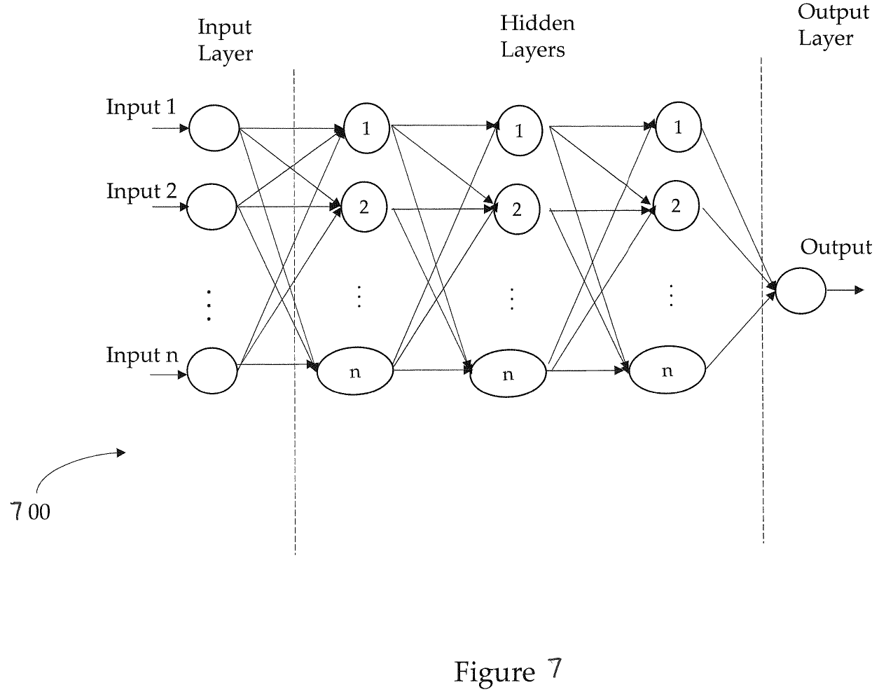

At operation 350, a machine learning (ML) model is trained. FIG. 7 schematically shows a machine learning model 700 for patient diagnostics in accordance with various embodiments. In various embodiments, operation 350 may be performed prior to any of operations 310, 320, 330 or 340.

In some embodiments, the process multiple learning models 700 may be trained, such as separate model each using parameters corresponding to different eye movements (e.g., one model for fixation, one model for saccades, and one model for smooth pursuit).

In some embodiments, the machine learning model is a neural network, as shown in FIG. 7. The machine learning model may also be based on another type of supervised machine learning model trained using patient test parameters. As shown in FIG. 7, the machine learning model includes an input layer having a plurality of inputs corresponding to the combination of patient test parameters. In some embodiments, some or all of the patient test parameters may be pre-processed before being input into the machine learning model. Among other things, some of the patient test parameters may be normalized. The machine learning model 700 also includes an output, which may be in the form of a score, a probability, or a classification, among other things.

The source of the training data may be patient test parameters for a set of healthy individuals. In some embodiments, the source of the training data may be patient test parameters for a combination of healthy and diagnosed individuals. For example, in order to quantify human smooth pursuit, a mathematical model is identified that describes its dynamics. In various embodiments, the smooth pursuit system may be considered as an unknown process that is driven by the visual stimulus input and producing an eye movement output.

Effectively, the mathematical model of smooth pursuit may be in the form:

$$y ( t ) = f ( u ( t ) , θ ) ,$$

where the output y(t) is produced by a function f of the input u(t) and model parameters θ. In the case of the human smooth pursuit system, y(t) may be considered to be the gaze and u(t) to be the visual stimulus. Both quantities being time-dependent makes f a model of a dynamical system.

For modeling, a model structure is considered for f that is suitable for describing the relationship between u(t) and y(t). Each considered model structure includes its own set of model parameters θ to be determined in order to obtain a model that is suitable. With the visual stimulus u(t) and ability to observe the gaze y(t) through the eyetracker, an individualized model of a person's smooth pursuit may be determined by finding a model structure and identifying θ in a way such that the model describes the empirical data. In various embodiments, this may be referred to as system identification.

In order compare models between different individuals, a model structure that is suitable for the population may be chosen and fixed. It is then possible to see how each individual's θ differs, as it may reveal unique qualities for different sub-groups of that population.

In various embodiments, a Volterra-Laguerre model, known to those skilled in the art, may be utilized as a black-box model suitable for complex systems that may present both linear and non-linear dynamics, which has been applied to modeling of the smooth pursuit system. In various embodiments, the input goes through a Laguerre filter layer, which divides the input into several components of an orthonormal basis. The outputs of the Laguerre filter layer are then used as input to a Volterra system model layer, which allows for modeling of smooth non-linear dynamics.

In various embodiments, Volterra-Laguerre models have the form:

$$y ( t ) = {y}_{0} H ( t ) + {Σ}_{n = 1}^{N} {Σ}_{{j}_{1} = 0}^{M} … {Σ}_{{j}_{n} = 0}^{M} {γ}_{n} ( {j}_{1} , … , {j}_{n} ) {ψ}_{{j}_{1}} ( t ) … {ψ}_{{j}_{n}} ( t ) ,$$

where H(t) is the Heaviside function, the ψ factors are the Laguerre filter output components, the model parameters θ include the constant offset factor y₀ and the Laguerre coefficients γ. The parameters M and N, referred to as the Laguerre order and the nonlinearity order, respectively, may be hyperparameters that determine the model complexity, as increasing those will increase the number of γ parameters. In some embodiments, system identification may follow the principle of Occam's razor, and select the least complex model that is sufficient for describing the system.

Choosing a highly complex model may allow for a perfect fit to an empirical data set, but those models tend to have poor generalizability when tested against a different data set of the same system, which may be referred to as overfitting. Another approach to avoid overfitting is to select a slightly more complex model structure than you expect that you will need for your system and use sparse estimation methods that are designed to prefer models using a smaller subset of the allowed parameters in θ, by penalizing models that use a larger number of parameters.

In some examples, M=N=2 may be used as a model structure. This may provide a θ vector with 10 elements. With this model structure selected, Volterra-Laguerre models for horizontal and vertical smooth pursuit may be estimated separately, giving a total of 20 parameters for the full 2D smooth pursuit. Using sparse estimation means that the final fully identified model may use significantly fewer than the 20 allowed parameters and the parameters that are ultimately selected may be different for different individuals.

However, eye motor control requires coordination of the extraocular muscles, with the superior/inferior rectus primarily controlling the vertical, the lateral/medial rectus primarily controlling the horizontal eye movements and the superior/interior oblique muscles controlling rotations in unison with the former four muscles. Capturing the horizontal-vertical cross-coupling of the eye motor control mechanisms would therefore allow the model to better describe the coordination of the extraocular muscles.

Accordingly, to include the horizontal-vertical cross-couplings in the model the stimulus and gaze may be described with complex numbers in accordance with the following:

$$u ( t ) = {u}_{x} ( t ) + i {u}_{y} ( t )$$

$$y ( t ) = {y}_{x} ( t ) + i {y}_{y} ( t ) ,$$

where subscripts x, y denote the horizontal and vertical component of the stimulus u(t) and the gaze y(t), and i=\sqrt{−1} is the imaginary number. A Volterra-Laguerre model structure may be employed, where the Laguerre filter outputs ψ and also the Laguerre coefficients γ are now complex-valued. In addition, the complex-valued VL model uses the complex conjugated Laguerre outputs ψ* to be able to capture all possible cross-couplings. Accordingly, the full, complex-valued VL model is in accordance with the following:

$$y ( t ) = {\underbrace{{y}_{0} H ( t )}}_{Constant term} + {Σ}_{j = 0}^{M} {\underbrace{[ {γ}_{1 , 0} ( j ) {ψ}_{j} ( t ) + {γ}_{0 , 1} ( j ) {ψ}_{j}^{*} ( t ) ]}}_{First order , linear terms} + {Σ}_{n = 2}^{N} \{ {Σ}_{{j}_{1} = 0}^{M} {Σ}_{{j}_{2} = {j}_{1}}^{M} … {Σ}_{{j}_{n} = {j}_{n - 1}}^{M} {\underbrace{[ {γ}_{n , 0} ( {j}_{1} , … , {j}_{n} ) {ψ}_{{j}_{1}} ( t ) … {ψ}_{{j}_{n}} ( t ) + {γ}_{0 , n} ( {j}_{1} , … , {j}_{n} ) {ψ}_{j 1}^{*} ( t ) … {ψ}_{{j}_{n}}^{*} ( t ) ]}}_{Higher order , nonlinear terms} + {Σ}_{m = 1}^{n - 1} [ {\underbrace{{Σ}_{{j}_{1} = 0}^{M} … {Σ}_{{j}_{n - m} = 0}^{M} {Σ}_{{l}_{1} = 0}^{M} … {Σ}_{{l}_{m} = 0}^{M} {γ}_{n - m , m} ( {j}_{1} , … , {j}_{n - m} , {l}_{1} , … , {l}_{m} ) {\prod }_{{k}_{1} = {j}_{1}}^{{j}_{n - m}} {ψ}_{{k}_{1}} ( t ) {\prod }_{{k}_{2} = {l}_{1}}^{{l}_{m}} {ψ}_{k 2}^{*} ( t )}}_{Higher order , nonlinear terms , mixing complex conjugate and non - conjugate terms} ] \} ,$$

where the Laguerre coefficients γ_{n,m} are used with subscripts n, m≥0 to denote the number of non-conjugated and conjugated Laguerre outputs are included, respectively, in each term. The total number of parameters for the complex VL model is

$$2 ( \begin{matrix}N + M + 1 \\ N\end{matrix} ) + {( M + 1 )}^{N} - 1 ,$$

which is significantly more than for the standard VL model. Therefore, sparse estimation methods that can identify a small subset of only the most significant parameters is essential. For comparison, the complex VL model with N=M=2 will have 28 parameters, whereas the standard VL model only had 20, and the model structure for this particular complex VL model is:

$$y ( t ) = {y}_{0} H ( t ) + {Σ}_{j = 0}^{2} [ {γ}_{1 , 0} ( j ) {ψ}_{j} ( t ) + {γ}_{0 , 1} ( j ) {ψ}_{j}^{*} ( t ) ] + {Σ}_{{j}_{1} = 0}^{2} {Σ}_{{j}_{2} = {j}_{1}}^{2} [ {γ}_{2 , 0} ( {j}_{1} , {j}_{2} ) {ψ}_{{j}_{1}} ( t ) {ψ}_{{j}_{2}} ( t ) + {γ}_{0 , 2} ( {j}_{1} , {j}_{2} ) {ψ}_{{j}_{1}}^{*} ( t ) {ψ}_{{j}_{2}}^{*} ( t ) ] + {Σ}_{{j}_{1} = 0}^{2} {Σ}_{{j}_{2} = 0}^{2} [ {γ}_{1 , 1} ( {j}_{1} , {j}_{2} ) {ψ}_{{j}_{1}} ( t ) {ψ}_{{j}_{2}}^{*} ( t ) ] .$$

Since the parameters γ gamma are complex-valued, they contain information about how much the horizontal and vertical eye movements are cross-coupled. This may be understood by observing the linear terms where the Laguerre outputs ψ describes horizontal stimuli as real-valued components and vertical stimuli as imaginary-valued components. The imaginary components of γ will therefore map horizontal stimulus to vertical gaze and vice versa. In addition, higher order terms also contain mappings of nonlinear combinations of horizontal and vertical stimuli.

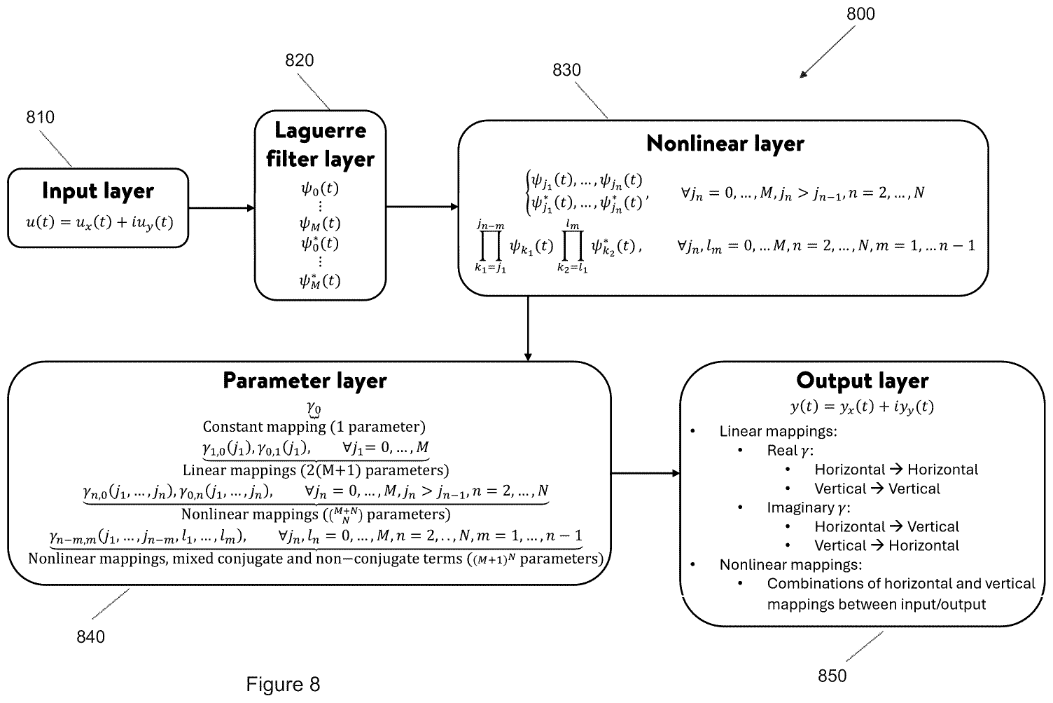

FIG. 8 shows an example modeling pipeline 800 for complex valued VL modeling of smooth pursuit in accordance with various embodiments. As shown in FIG. 8, the modeling pipeline includes an input layer 810, a Laguerre filter layer 820, a nonlinear layer 830, a parameter layer 840 and an output layer 850, described in more detail below.

In various embodiments, the input stimuli is encoded as a complex variable in the input layer 810. The input is processed by the Laguerre filter layer 820, producing the 2(M+1) Laguerre filter outputs ψ and their complex conjugates ψ*. The Laguerre filter outputs are then combined, producing

$$( \begin{matrix}M + N \\ N\end{matrix} )$$

nonlinear terms (830), where conjugate and non-conjugate terms are not mixed, and (M+1)^{N} nonlinear terms where conjugate and non-conjugate terms are mixed. The order of the nonlinear terms is set by N, where N=2 produces quadratic terms, N=3 produces cubic terms, and so on.

Next, the parameters y are identified as shown in the parameter layer 840. In various embodiments, the VL model, and the complex VL model in particular, tends to be overparametrized, (e.g., the model structure allows a larger number of parameters that is necessary to map the input to the output data). This may be handled using sparse estimation methods, which are designed to identify only a smaller subset of significant parameters that captures the input-output relationships. Finally, the set of identified parameters can be analyzed in terms of how they map the input stimuli to the output gaze data. Specifically, the linear mappings reveal the direct couplings in their real-valued components and the cross-coupling between horizontal and vertical stimuli/gaze in their imaginary-valued components.

The nonlinear mappings reveal if higher orders of complexities are present, since these terms combine both horizontal and vertical components of the Laguerre outputs at the output layer 850. For example, the linear mappings may show the real and imaginary mappings for horizontal to vertical and vertical to horizontal as well as the nonlinear mappings (e.g., combinations of horizontal and vertical mappings between an input and output).

The sum of the parameter extraction and modeling described above provides a set of quantifications of how a person's eye movements have responded to the visual stimulus. At operation 370, then the patient diagnostic parameters may be determined. In various embodiments, the patient diagnostic parameters may include a probability that the patient has the disease for which the patient is being evaluated. In addition, the patient diagnostic parameters may include an objective indicator of the severity of the disease for the patient, the probability, severity, and/or presence of a disease symptom. In various embodiments, the eye movements are therefore correlated to a neurological condition (e.g., Parkinson's). In various embodiments, the objective indicator may include a score (e.g., 1-10).

Accordingly, the collected quantifications may be used as a biomarker for predicting clinically significant changes in disease state, presence of motor or non-motor symptoms or treatment response. At operation 380 then, patient diagnostic parameters are output for a treatment plan or parameter.

In various embodiments, the treatment plan or parameter may include a probability that a particular treatment will be effective, a score, or a dosage, among other things.

The treatment parameter may also correspond to a medication, a therapy, a dosage, or a physical device setting, among other things. The treatment plan may include administering a new medication or an updated dosage, administering a new therapy or adjusted therapy, or providing a physical device, or adjusting a physical device setting. For example, the treatment plan may correspond to a deep brain stimulator device or a new electrical signal for an existing deep brain stimulator. In some embodiments, the method 300 may be form a feedback loop useful for tuning a treatment, such as a deep brain stimulator, for example.

The operations of FIG. 3 are merely illustrative, and variations are contemplated to be within the scope of the present disclosure. In embodiments, the operations may include other operations not illustrated in FIG. 3. In embodiments, the operations may not include every operation illustrated in FIG. 3. In embodiments, the operations may be implemented in a different order than that illustrated in FIG. 3. Such and other embodiments are contemplated to be within the scope of the present disclosure. Persons of skill in the art will appreciate that, although various example components are described as perform various functions, other components may perform those functions described in FIG. 3.

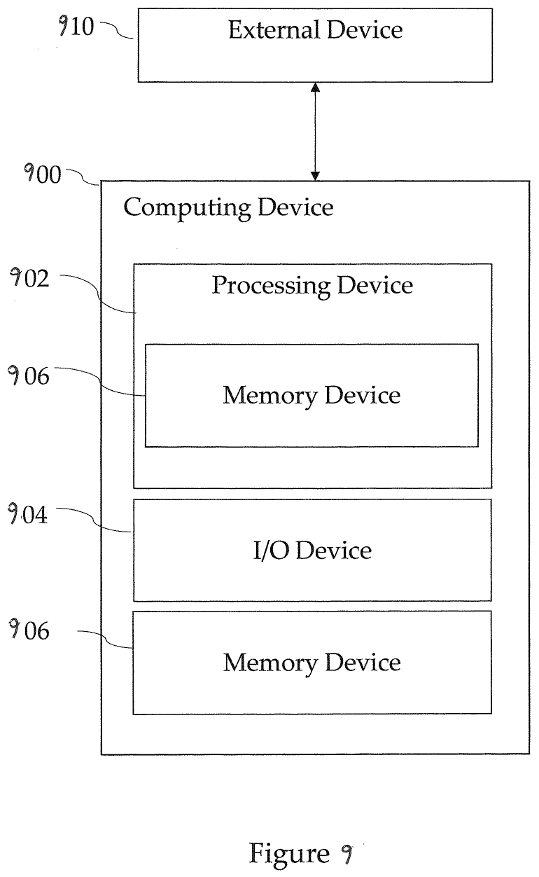

FIG. 9 schematically shows a computing device 900 in accordance with various embodiments. The computing device 900 is one example of one of the devices in the eye tracking system 120 a computing device which is used to perform one or more operations of process 200 illustrated in FIG. 2. The computing device 900 includes a processing device 902, an input/output device 904, and a memory device 906. The computing device 900 may be a stand-alone device, an embedded system, or a plurality of devices configured to perform the functions described with respect to one of the components of power network 100. Furthermore, the computing device 900 may communicate with one or more external devices 910.

The input/output device 904 enables the computing device 900 to communicate with an external device 910. For example, the input/output device 904 may be a network adapter, a network credential, an interface, or a port (e.g., a USB port, serial port, parallel port, an analog port, a digital port, VGA, DVI, HDMI, FireWire, CAT 5, Ethernet, fiber, or any other type of port or interface), among other things. The input/output device 904 may be comprised of hardware, software, or firmware. The input/output device 904 may have more than one of these adapters, credentials, interfaces, or ports, such as a first port for receiving data and a second port for transmitting data, among other things.

The external device 910 may be any type of device that allows data to be input or output from the computing device 900. For example, the external device 910 may be a meter, a control system, a sensor, a mobile device, a reader device, equipment, a handheld computer, a diagnostic tool, a controller, a computer, a server, a printer, a display, a visual indicator, a keyboard, a mouse, or a touch screen display, among other things. Furthermore, the external device 910 may be integrated into the computing device 900. More than one external device may be in communication with the computing device 900.

The processing device 902 may be a programmable type, a dedicated, hardwired state machine, or a combination thereof. The processing device 902 may further include multiple processors, Arithmetic-Logic Units (ALUs), Central Processing Units (CPUs), Digital Signal Processors (DSPs), or Field-programmable Gate Arrays (FPGA), among other things. For forms of the processing device 902 with multiple processing units, distributed, pipelined, or parallel processing may be used. The processing device 902 may be dedicated to performance of just the operations described herein or may be used in one or more additional applications. The processing device 902 may be of a programmable variety that executes processes and processes data in accordance with programming instructions (such as software or firmware) stored in the memory device 906. Alternatively or additionally, programming instructions are at least partially defined by hardwired logic or other hardware. The processing device 902 may be comprised of one or more components of any type suitable to process the signals received from the input/output device 904 or elsewhere, and provide desired output signals. Such components may include digital circuitry, analog circuitry, or a combination thereof.

The memory device 906 in different embodiments may be of one or more types, such as a solid-state variety, electromagnetic variety, optical variety, or a combination of these forms, to name but a few examples. Furthermore, the memory device 906 may be volatile, nonvolatile, transitory, non-transitory or a combination of these types, and some or all of the memory device 906 may be of a portable variety, such as a disk, tape, memory stick, or cartridge, to name but a few examples. In addition, the memory device 906 may store data which is manipulated by the processing device 902, such as data representative of signals received from or sent to the input/output device 904 in addition to or in lieu of storing programming instructions, among other things. As shown in FIG. 9, the memory device 906 may be included with the processing device 902 or coupled to the processing device 902, but need not be included with both.

It is contemplated that the various aspects, features, processes, and operations from the various embodiments may be used in any of the other embodiments unless expressly stated to the contrary. Certain operations illustrated may be implemented by a computer executing a computer program product on a non-transient, computer-readable storage medium, where the computer program product includes instructions causing the computer to execute one or more of the operations, or to issue commands to other devices to execute one or more operations.

While the present disclosure has been illustrated and described in detail in the drawings and foregoing description, the same is to be considered as illustrative and not restrictive in character, it being understood that only certain exemplary embodiments have been shown and described, and that all changes and modifications that come within the spirit of the present disclosure are desired to be protected. It should be understood that while the use of words such as “preferable,” “preferably,” “preferred” or “more preferred” utilized in the description above indicate that the feature so described may be more desirable, it nonetheless may not be necessary, and embodiments lacking the same may be contemplated as within the scope of the present disclosure, the scope being defined by the claims that follow. In reading the claims, it is intended that when words such as “a,” “an,” “at least one,” or “at least one portion” are used there is no intention to limit the claim to only one item unless specifically stated to the contrary in the claim. The term “of” may connote an association with, or a connection to, another item, as well as a belonging to, or a connection with, the other item as informed by the context in which it is used. The terms “coupled to,” “coupled with” and the like include indirect connection and coupling, and further include but do not require a direct coupling or connection unless expressly indicated to the contrary. When the language “at least a portion” or “a portion” is used, the item can include a portion or the entire item unless specifically stated to the contrary. Unless stated explicitly to the contrary, the terms “or” and “and/or” in a list of two or more list items may connote an individual list item, or a combination of list items. Unless stated explicitly to the contrary, the transitional term “having” is open-ended terminology, bearing the same meaning as the transitional term “comprising.”

Various embodiments of the invention may be implemented at least in part in any conventional computer programming language. For example, some embodiments may be implemented in a procedural programming language (e.g., “C”), or in an object-oriented programming language (e.g., “C++”). Other embodiments of the invention may be implemented as a pre-configured, stand-alone hardware element and/or as preprogrammed hardware elements (e.g., application specific integrated circuits, FPGAS, and digital signal processors), or other related components.

In an alternative embodiment, the disclosed apparatus and methods (e.g., see the various flow charts described above) may be implemented as a computer program product for use with a computer system. Such implementation may include a series of computer instructions fixed either on a tangible, non-transitory medium, such as a computer readable medium (e.g., a diskette, CD-ROM, ROM, or fixed disk). The series of computer instructions can embody all or part of the functionality previously described herein with respect to the system.

Those skilled in the art should appreciate that such computer instructions can be written in a number of programming languages for use with many computer architectures or operating systems. Furthermore, such instructions may be stored in any memory device, such as semiconductor, magnetic, optical or other memory devices, and may be transmitted using any communications technology, such as optical, infrared, microwave, or other transmission technologies.

Among other ways, such a computer program product may be distributed as a removable medium with accompanying printed or electronic documentation (e.g., shrink wrapped software), preloaded with a computer system (e.g., on system ROM or fixed disk), or distributed from a server or electronic bulletin board over the network (e.g., the Internet or World Wide Web). In fact, some embodiments may be implemented in a software-as-a-service model (“SAAS”) or cloud computing model. Of course, some embodiments of the invention may be implemented as a combination of both software (e.g., a computer program product) and hardware. Still other embodiments of the invention are implemented as entirely hardware, or entirely software.

The embodiments of the invention described above are intended to be merely exemplary; numerous variations and modifications will be apparent to those skilled in the art. Such variations and modifications are intended to be within the scope of the present invention as defined by any of the appended claims. It shall nevertheless be understood that no limitation of the scope of the present disclosure is hereby created, and that the present disclosure includes and protects such alterations, modifications, and further applications of the exemplary embodiments as would occur to one skilled in the art with the benefit of the present disclosure.

1. A method of managing a neurological condition, the method comprising:
displaying a test target;
detecting, by an image capture device, movement of a patient eye with respect to the test target;
receiving data relating to the movement of the patient eye as an input to a machine learning model;
correlating the data relating to the movement of the patient eye with respect to the test target to a neurological condition; and
producing objective output information by the machine learning model based on the correlation of the data to the neurological condition indicating a level of progression of the neurological condition.

2. The method of claim 1, wherein the detecting movement is performed during one or more eye movement tests.

3. The method of claim 2, wherein the one or more eye movement tests comprises one or more of a fixation test or a smooth pursuit test.

4. The method of claim 3, wherein the fixation test further comprises displaying the test target at one or more locations and detecting a latency, duration, peak amplitude, or peak velocity of the movement of the patient eye.

5. The method of claim 4, further comprising detecting a catch-up saccade of the movement of the patient eye.

6. The method of claim 2, wherein the smooth pursuit test comprises moving the test target along a trajectory on the display and detecting the movement of the patient eye following the trajectory.

7. The method of claim 1, further comprising training a machine learning model using the data relating to the movement of the patient eye during the eye movement test.

8. The method of claim 7, further comprising outputting a patient treatment parameter based upon the correlation of the data relating to the movement of the patient eye to a neurological condition.

9. The method of claim 1, further comprising planning a treatment for the neurological disorder based on the correlation of the data to the neurological condition indicating the level of progression of the neurological condition.

10. A system, comprising:
an image capture device configured to detect movement of a patient eye with respect to a displayed test target; and
a processor in communication with the image capture device, the processor configured to receive, from the image capture device, data relating to the movement of the patient eye as an input to a machine learning model, correlate the data relating to the movement of the patient eye with respect to the test target to a neurological condition, and produce objective output information by the machine learning model based on the correlation of the data to the neurological condition indicating a level of progression of the neurological condition.

11. The system of claim 10, wherein the image capture device detects movement during one or more eye movement tests.

12. The system of claim 11, wherein the one or more eye movement tests comprises one or more of a fixation test or a smooth pursuit test.

13. The system of claim 12, wherein the fixation test further comprises displaying the test target on the display at one or more locations on the display and detecting a latency, duration, peak amplitude, or peak velocity of the movement of the patient eye.

14. The system of claim 13, wherein the image capture device detects a catch-up saccade of the movement of the patient eye.

15. The system of claim 12, wherein the smooth pursuit test comprises moving the test target along a trajectory on the display and detecting the movement of the patient eye following the trajectory.

16. The system of claim 10, wherein the processor is further configured to train a machine learning model using the data relating to the movement of the patient eye during the eye movement test.

17. The system of claim 16, wherein the processor is further configured to output a patient treatment parameter based upon the correlation of the data relating to the movement of the patient eye to a neurological condition.

18. The system of claim 10, wherein the processor is further configured to output a plan for a treatment for the neurological disorder based on the correlation of the data to the neurological condition indicating the level of progression of the neurological condition.

19. A computer program product for use on a computer system, the computer program product comprising a tangible, non-transient computer usable medium having computer readable program code thereon, the computer readable program code comprising:
program code for displaying a test target;
program code for receiving data relating to a movement of a patient eye with respect to the test target as an input to a machine learning model;
program code for correlating the data relating to the movement of the patient eye with respect to the test target to a neurological condition; and
program code for producing objective output information by the machine learning model based on the correlation of the data to the neurological condition indicating a level of progression of the neurological condition.

20. The computer program product of claim 19, further comprising program code for outputting a patient treatment parameter based upon the correlation of the data relating to the movement of the patient eye to a neurological condition.
